(19) United States
(12) Patent Application Publication
(10) Pub. No.: US 2016/0346231 A1
(43) Pub. Date: Dec. 1, 2016

(54) Disulfiram Compositions and Treatments for Brain Tumors

(71) Applicant: TEXAS TECH UNIVERSITY SYSTEM, Lubbock, TX (US)

(72) Inventors: Kalkunte S. Srivenugopal, Amarillo, TX (US); Ameya M. Paranjpe, Amarillo, TX (US)

(21) Appl. No.: 15/116,757

(22) PCT Filed: Feb. 6, 2015
(86) PCT No.: PCT/US2015/014789

§ 371 Date: Aug. 4, 2016

Related U.S. Application Data
(60) Provisional application No. 61/936,455, filed on Feb. 6, 2014.

(51) Int. Cl. A61K 31/145; A61K 45/06; A61K 31/621

(52) U.S. Cl. CPC A61K 31/145; A61K 31/621; A61K 45/06

(57) ABSTRACT

The present disclosure provides a compositions and methods of inhibiting 06-methylguanine DNA methyltransferase in human tumor cells by providing an effective amount of a disulfiram composition to inhibit the 06-methylguanine DNA methyltransferase in a pharmaceutically acceptable carrier to inhibit 06-methylguanine DNA methyltransferase.

### TECHNICAL FIELD OF THE INVENTION

The present invention relates in general to the field of methods of treating cancer, specifically to disulfiram compositions and methods of making and using analogues for the treatment of cancer.

### BACKGROUND OF THE INVENTION

Without limiting the scope of the invention, its background is described in connection with methods of treating cancer. The incidence of brain tumors in adults and pediatric patients has seen a continued increase in recent years with an estimated 17,000 new cases diagnosed and 12,000 deaths every year in the United States. Brain tumors are also the second leading cause of cancer deaths among pediatric patients. Gliomas, medulloblastomas, and other brain cancers remain the most therapeutically challenging and chemotherapy using hydrophobic alkylating agents that cross the blood-brain barrier is a mainstay in their treatment. The clinically used alkylating agents also sensitize the brain tumors to radiation and therefore, there is a great need for strengthening/improving the efficacy of the monofunctional and bifunctional alkylating drugs used for central nervous system cancers. Disulfiram (DSF) has been shown to increase the cytotoxicity of many anticancer drugs such as the cisplatin, gemcitabine, paclitaxel and 5-fluorouracil.

O⁶-methylguanine DNA methyltransferase (MGMT) is a DNA repair protein expressed variably in human tissues and functions to protect the genome against mutations from the alkylating agents of endogenous and environmental origins. In up to 80% of brain cancers, it is highly expressed and its repair function interferes with the cytotoxic actions of the alkylating agents (nitrosoureas, temozolomide etc) used in the chemotherapy. This is because, MGMT scavenges the alkyl groups introduced into the DNA by the drugs and nullifies the tumor cell killing. There are very few inhibitors for MGMT, and the one in clinical trials causes prolonged inhibition of DNA repair, and this has not been useful for successful therapy as explained in the narrative above. Disulfiram is expected to overcome this problem. An inhibitor of MGMT called 06-benzylguanine (BG) went into clinical trials about 10 years ago, to inhibit all of MGMT in tumors and then give the alkylating drugs to patients to introduce more damage to the DNA, and thus eliminate the tumor cells in the brain. However, BG suppresses the MGMT activity in both normal and tumor tissues for a long time, and this suppression in the bone marrow stem cells causes greater damage in them, leading to deficient production of blood cells, and discontinuance of therapy.

U.S. Pat. No. 6,548,540, entitled, “Method of treating cancer using dithiocarbamate derivatives,” discloses dithiocarbamate, particularly tetraethylthiuram disulfide, and thiocarbamate anions strongly inhibit the growth of cancer cells of a variety of cell types. Such inhibitory effect is enhanced by heavy metal ions such as copper ions, cytokines and ceruloplasmin and a method is presented for using tetraethylthiuram disulfide to reduce tumor growth, and to potentiate the effect of other anticancer agents. The entire contents of which are incorporated herein by reference.

U.S. Pat. No. 6,288,110, entitled, “Pharmaceutical compositions comprising disulfiram,” discloses disulfiram (tetraethylthiuram disulfide) to inhibit angiogenesis and to be useful in the treatment of angiogenesis-dependent disorders, including neoplasms, and to prevent cell hyperproliferation and formation of clots along or around medical devices. The entire contents of which are incorporated herein by reference.

### SUMMARY OF THE INVENTION

The present disclosure provides a method of inhibiting O⁶-methylguanine DNA methyltransferase in human tumor cells by providing a subject having primary or metastatic cancers; providing an effective amount of a disulfiram composition to inhibit an O⁶-methylguanine DNA methyltransferase in a pharmaceutically acceptable carrier to the tumors; and inhibiting a O6-methylguanine DNA methyltransferase with the disulfiram. The disulfiram composition may be administered in a dosage of between about 125 to about 1000 mg per day of body weight.

The disulfiram composition may be disulfiram, copper-chelated disulfiram, copper-chloride disulfiram or a combination thereof. The disulfiram composition may be administered parenterally, orally or both. The disulfiram composition may be administered in combination with a metal chelate that includes an ion selected from the group consisting of arsenic, bismuth, cobalt, copper chromium, gallium, gold iron, manganese, nickel, silver, titanium, vanadium, selenium and zinc.

The human tumor cells may be human brain tumors or fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

The composition may further include a cytotoxic agent, e.g., cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin, rapamycin and its derivatives, sirolimus, temsirolimus, everolimus, zotarolimus and deforolimus. Also included in the definition are microtubulin inhibitors include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a,-9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-py-razolo[4,5,1-de]acridin-6-one, N-[1-[2(diethylamino)ethylamino]-7-methoxy-9-oxo-9H-thioxanthen-4-ylmethy-1]formamide, N-(2-(dimethylamino)ethyl)acrid-ine-4-carboxamide, and 6-[[2-(dimethylamino)ethyl]amino]-3-hydroxy-7H-indeno[2-,1-c]quinolin-7-one.

The present disclosure also provides a method of inhibiting O⁶-methylguanine DNA methyltransferase in human tumor cells by providing a subject having cancer; providing an effective amount of a sulfhydryl group-conjugating agent to inhibit an O⁶-methylguanine DNA methyltransferase in a pharmaceutically acceptable carrier, and inhibiting O⁶-methylguanine DNA methyltransferase through cysteine-conjugation.

The present disclosure provides a chemotherapy composition comprising effective amount of a SH (cysteine)-conjugating agent to inhibit an O⁶-methylguanine DNA methyltransferase disposed in a pharmaceutically acceptable carrier; and one or more cytotoxic agents disposed in a pharmaceutically acceptable carrier.

The present disclosure provides a method of inhibiting DNA repair in subject suffering from a proliferation disorder by providing a subject suffering from a proliferation disorder; providing an effective amount of a cytotoxic agents and a disulfiram composition, a SH-conjugating agent in a pharmaceutically acceptable carrier, or both to inhibit an O⁶-methylguanine DNA methyltransferase and increasing the anticancer efficacy of alkylating drugs.

The present disclosure provides a method of treating a proliferation disorder by providing a subject having one or more tumors; providing an effective amount of a disulfiram composition to inhibit an O⁶-methylguanine DNA methyltransferase in a pharmaceutically acceptable carrier to the one or more tumors; and inhibiting a O⁶-methylguanine DNA methyltransferase with the disulfiram.

One embodiment includes a pharmaceutical composition comprising effective amount of a disulfiram composition in a pharmaceutically acceptable carrier for use in the treatment of tumor cells wherein the disulfiram composition inhibits an O⁶-methylguanine DNA methyltransferase in the tumor cells.

One embodiment includes a pharmaceutical composition comprising an effective amount of a nitrosylating agent in a pharmaceutically acceptable carrier to inhibit O⁶-methylguanine DNA methyltransferase in tumor cells.

One embodiment includes a chemotherapy composition comprising effective amount of a disulfiram composition to inhibit an O⁶-methylguanine DNA methyltransferase disposed in a pharmaceutically acceptable carrier; and one or more cytotoxic agents disposed in a pharmaceutically acceptable carrier.

One embodiment includes a chemotherapy composition comprising effective amount of a nitrosylating agent to inhibit an O⁶-methylguanine DNA methyltransferase disposed in a pharmaceutically acceptable carrier; and one or more cytotoxic agents disposed in a pharmaceutically acceptable carrier.

One embodiment includes a pharmaceutical composition for inhibiting DNA repair in subject suffering from a proliferation disorder, wherein the pharmaceutical composition comprises an effective amount of a cytotoxic agents and a disulfiram composition, a nitrosylating agent or both in a pharmaceutically acceptable carrier to inhibit an O⁶-methylguanine DNA methyltransferase.

In any of the embodiments disclosed herein the disulfiram composition may be disulfiram, copper-chelated disulfiram, copper-chloride disulfiram or a combination thereof. In any of the embodiments disclosed herein a disulfiram composition may be administered in combination with a metal chelate that includes an ion selected from the group consisting of arsenic, bismuth, cobalt, copper chromium, gallium, gold iron, manganese, nickel, silver, titanium, vanadium, selenium and zinc. In any of the embodiments disclosed herein the nitrosylating agent may be nitroaspirin. In any of the embodiments disclosed herein the pharmaceutical composition may be administered in a dosage of between about 125 to about 1000 mg per day of body weight.

### DESCRIPTION OF THE DRAWINGS

For a more complete understanding of the features and advantages of the present invention, reference is now made to the detailed description of the invention along with the accompanying figures and in which:

FIGS. 1A-1D show concentration-dependent inhibition of MGMT activity and MGMT degradation induced by DSF and its derivatives in human brain tumor cells. FIG. 1A shows inhibition of the DNA repair activity of purified recombinant MGMT (rMGMT) and cellular MGMT by increasing DSF concentrations. rMGMT or UW228 cell-free extracts were first treated with DSF at concentrations shown for 20 min at room temperature followed by the addition of DNA substrate. FIG. 1B shows inhibition of MGMT activity and loss of MGMT protein in T98G and UW228 cells. FIG. 1C shows the effect of CuDSF on MGMT activity and protein levels in UW228 and T98G cells. FIG. 1D shows the effect of copper and DSF treatments on MGMT.

FIGS. 2A-2C show time-dependent elimination of MGMT protein in brain tumor cells by DSF, CuDSF and Cu+DSF. FIG. 2A shows T98G and UW228 cells were treated 50 μM DSF. At times indicated, MGMT activity and protein levels were assessed. FIG. 2B shows potent inactivation of MGMT by 10 μM CuDSF in T98G and UW228 cells. MGMT Protein levels in drug-treated UW228 cells are shown. FIG. 2C shows inhibition of MGMT in tumor cells after a combined treatment of 0.1 μM CuCl2 and DSF.

FIG. 3A shows Immunoflourecence decreased MGMT protein levels in DSF-treated UW228 cells. Drug treatments and exposure times are shown in labels. FIG. 3B shows depletion and repletion kinetics of MGMT protein following BG and DSF treatments. FIG. 3C shows that Cys145 of MGMT is the target site for DSF. FIG. 3D shows DSF abrogates the binding of the BG probe to cellular MGMT. UW228 cell extracts proficient in MGMT were incubated with DSF or N-ethylmaleimide (0.5 mM) for 20 min followed by BG-PEG-biotin as described for the purified proteins.

FIGS. 4A-4E show proteasomal degradation of DSF modified MGMT. FIG. 4A shows in vitro degradation of DSF treated rMGMT protein. FIG. 4B shows the proteasome inhibitor PS-341 curtails the degradation of DSF-modified MGMT protein in brain tumor cells. FIG. 4C shows DSF increases the DNA damage induced by MGMT-targeted alkylating agents. FIG. 4D shows G2/M blockade induced by BCNU is enhanced and extended in the presence of DSF. Histograms showing results of FACS-based cell cycle analysis of UW228 cells treated with BCNU alone, DSF alone, and BCNU+DSF combination. Asynchronous cultures were treated or untreated with DSF (50 μM for 12 h) and then incubated with 100 μM BCNU. The cells were trypsinized at times indicated and analyzed by flow cytometry. FIG. 4E shows enhanced levels of apoptosis markers—cleaved caspase 3 and cleaved PARP after DSF+BCNU treatments compared with BCNU alone.

FIGS. 5A-5G show DSF preexposure sensitizes the brain tumor cells to the clinically used alkylating agents. FIG. 5A shows cell survival after DSF treatment alone. UW228 cells were treated with increasing concentrations of DSF (1-100 μM) for 24 hours. FIG. 5B shows TMZ mediated cell killing with and without DSF preexposure in UW228 cells. FIG. 5C shows BCNU mediated cell killing with and without DSF preexposure in UW228 cells. FIG. 5D shows DSF did not increase the cytotoxicity of melphalan, an N7, but not an O6-alkylator of guanine in UW228 cells. FIG. 5E shows DSF did not potentiate the TMZ-induced cytotoxicity in the MGMT-deficient U87 malignant glioma cells. FIG. 5F shows DSF did not potentiate BCNU-induced cell killing in the MGMT-deficient U87 cells. In all these cases, the tumor cells were treated or untreated with 50 μM DSF for 12 hours followed by the alkylating drugs for 4 days before the MTT assays. FIG. 5G shows soft agar colony formation assay for cell survival analysis.

FIGS. 6A-6C show significant downregulation of MGMT activity and protein in mouse brain and liver tissues after DSF (150 mg/kg) administration. FIG. 6A shows DSF treatment resulted in a sustained 40% inhibition of brain MGMT activity starting at 9 hours (upper panel). Western blot analysis (bottom panel) shows a gradual decrease of MGMT protein in the same time course. FIG. 6B shows decreased hepatic MGMT activity (60% inhibition, upper panel) and protein levels (lower panel) after DSF administration. FIG. 6C shows the consequences of MGMT inhibition by DSF and increased in tumor sensitivity to alkylating agents.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the abbreviation DSF denotes 1,1′,1″,1′″-[disulfanediylbis(carbonothioylnitrilo)]tetraethane called disulfiram which is an FDA approved drug used as alcohol abuse deterrent based on the inhibitory activity on aldehyde dehydrogenase having the structure:

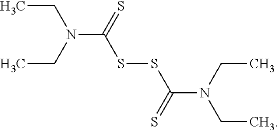 [SMILES: CCN(CC)C(=S)SSC(=S)N(CC)CC]

It undergoes thiol-disulfide exchange, also known as S-thiolation, targeting specific protein sulfhydryl groups and the critical cysteine residues in proteins. If another cysteine exists in the vicinity, intramolecular S—S bonds can result.

As used herein the abbreviation CuDSF denotes copper chelated disulfiram.

As used herein the abbreviation BG denotes O⁶-benzylguanine.

As used herein the abbreviation BCNU denotes 1,3-bis-2-chloroethylnitrosourea.

As used herein the abbreviation TMZ denotes temozolomide.

As used herein the abbreviation MGMT denotes O⁶-methylguanine DNA methyltransferase.

As used herein the abbreviation PS341 denotes bortezomib.

As used herein the abbreviation rMGMT denotes recombinant MGMT protein.

As used herein the abbreviation ALDH denotes aldehyde dehydrogenase.

As used herein the abbreviation ub denotes ubiquitin.

As used herein the abbreviation PARP denotes poly ADP-ribose polymerase.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

As used herein the term “Treatment” denotes an intervention performed with the intention of preventing the development or altering the pathology or symptoms of a disorder and refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

As used herein the term “dosing” and “treatment” denotes any process, action, application, therapy or the like, wherein a subject, particularly a human being, is rendered medical aid with the object of improving the subject's condition, either directly or indirectly.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human); and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein the term “Cytotoxic agents” denote compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

The present invention fulfills an urgent need for inhibiting the DNA repair protein MGMT (O6-methylguanine-DNA methyltransferase) and improving the efficacy of alkylating agents. MGMT is highly expressed in human cancers and prevents the formation of cytotoxic lesions in alkylated DNA. Current clinical trials involving MGMT depletion by O⁶-benzylguanine (BG), although promising, are beset with severe toxicity to the bone marrow, which has necessitated the transduction of BG-resistant MGMT gene into hematopoietic stem cells.

The present disclosure provides disulfiram as a direct and mechanism-based inhibitor of the human DNA repair protein MGMT. The present disclosure provides a composition that curtails MGMT for a shorter time and still be able incur sufficient damage in the tumor DNA. The present disclosure provides disulfiram, a FDA-approved drug for treatment of alcoholism, is an excellent inhibitor of MGMT.

Disulfiram binds with a critical cysteine residue at the active site of MGMT and turns off DNA repair. Disulfiram has the ability to penetrate the blood brain barrier, and we have shown that it strongly inhibits MGMT in brain tumor cells and mouse brain. The failure of chemotherapy and not being able to improve the quality of life in pediatric and adult patients with brain tumors is largely due to the overexpression of MGMT. Disulfiram is very safe (in people not drinking alcohol), does not induce any toxicity and can be given in large amounts orally to enable sustained availability to the brain. The pharmacology, dose, adverse effects and safety issues for disulfiram have been very well worked out.

There are very few inhibitors for MGMT, and the one in clinical trials caused prolonged inhibition of DNA repair, which has not been useful for successful therapy. Disulfiram as disclosed herein overcomes this problem.

The present disclosure provides disulfiram compositions to affect human MGMT, which removes the mutagenic O⁶-akyl groups from guanines, and thus renders the normal tissues and brain tumors resistant to alkylation DNA damage. The present disclosure provides DSF, copper-chelated DSF (CuDSF) or CuCl2-DSF combination as treatments to inhibit the MGMT activity in two brain tumor cell lines in a rapid and dose dependent manner. The drug treatments resulted in the loss of MGMT protein from tumor cells and the degradation occurred through the ubiquitin-proteasome pathway. Evidence showed that Cys145, a reactive cysteine, critical for DNA repair was the sole site of DSF modification in the MGMT protein. DSF was a weaker inhibitor of MGMT, compared to the established O⁶-benzylguanine, nevertheless, the 24-36 hours suppression of MGMT activity in cell cultures vastly increased the alkylation-induced DNA interstrand crosslinking, G2/M cell cycle blockade, cytotoxicity and the levels of apoptotic markers. Mice treated with DSF showed significantly attenuated levels of MGMT activity and protein in the liver and brain tissues. The present disclosure provides strong and direct inhibition of MGMT-mediated DNA repair by the nontoxic DSF and support the repurposing of this brain penetrating drug for glioma therapy. Cysteine 145 of MGMT accepts the alkyl groups for pharmacological intervention. Cys145 has a pKa of 4.8 due to its microenvironment and is susceptible for glutathionylation and nitrosylation. S-Thiolation and S-nitrosylation are reversible posttranslational mechanisms that gauge the intracellular redox and transduce them into functional responses.

The present disclosure provides two nontoxic small molecules which readily react with reactive cysteines, namely, the NCX-4016 (nitro-aspirin capable of S-nitrosylation) and disulfiram (capable of thiol-conjugation).

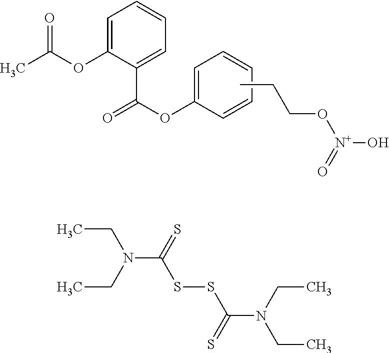

NCX-4016, also called a fatty aspirin, is degraded by plasma and tissue esterases to release NO in a sustained manner. In three MGMT-proficient human cancer cell lines (HT29, T47D, and HCT116), nitro-aspirin at very low concentrations (5-10 μM) caused a 90% inhibition of MGMT activity within 1 hour of exposure. Interestingly, the MGMT protein also disappeared with similar kinetics after NCX-4016 treatment; approx. 80-90% of MGMT was degraded after 5 μM NO-aspirin treatment for 2 hours. These data are highly comparable or better than those reported for BG. Further, purified MGMT or tumor cell extracts exposed to NCX-4016 failed to bind the biotin-labeled BG, indicating Cys145 to be the site of nitrosylation. More than 60% of MGMT protein was regenerated at 24 hours when NO-aspirin treated HT29 cells were post-incubated in drug-free medium, indicating a transient inhibition and rapid repletion. Disulfiram (DS), the alcohol deterrent drug, also curtailed MGMT activity in HT29 and HCT116 cells with a 20 hours 400 μM treatment causing a 95% inhibition. Disulfiram at 200 μM induced about 70% degradation of MGMT at 12 hours. Other redox-sensitive proteins such as the wild-type and mutant p53, NF-κB, and ubiquitin E1 were all degraded by disulfiram in a dose-dependent manner. In mice a single injection (100 mg/kg) of NCX-4016 causes 50-70% inhibition of MGMT activity in mice brain and liver. Because NO-aspirin, is non-toxic (IC50>500 μM for cell lines), yields a chemopreventive by-product, unlikely to elicit tumor resistance, and is lipophilic enough to cross the blood brain barrier (BBB), as such it can be well exploited for glioma therapy. The redox-regulated proteins are ‘druggable’ and highlight options for redox-driven therapeutic strategies.

O⁶-Methylguanine DNA-methyltransferase (MGMT) is a unique antimutagenic DNA repair protein that plays a crucial role in the defense against alkylating agents, particularly, those that generate the O⁶-alkylguanines. Guanine is the most preferred base for alkylation, and the adducts at the O⁶-guanine are particularly critical, because, the O⁶-alkylguanines pair aberrantly with thymine, resulting in GC to AT transitions. MGMT repairs O⁶-alkylguanine and O⁴-alkylthymine lesions by transferring the alkyl groups to an active site cysteine residue (Cys145) in the protein in a stoichiometric and suicidal reaction, so that the guanine in the DNA is simply restored in an error-free direct reversal reaction. Because, the alkyl group is covalently bound to the protein, MGMT is functionally inactivated after each reaction, and the inactive protein is degraded through the ubiquitin proteolytic pathway. MGMT is abundantly expressed in liver and other normal tissues, but is present at very low levels in the bone marrow and normal brain. The repair function of MGMT is essential for the removal of O⁶-guanine alkylations introduced by the carcinogens present in cooked meat, endogenous metabolites such as the S-adenosylmethionine, nitrosated amino acids and tobacco smoke, and maintaining genomic stability. MGMT appears to have a strong linkage with another public health problem, namely, the chronic alcohol abuse and the resulting pathological effects in liver and brain as well. A number of studies have described the suppression of MGMT and an increased alkylation damage following acute or chronic alcohol intake. Disulfiram (DSF, bis-diethylthiocarbamoyl disulfide), also known as Antabuse, is a carbamate derivative clinically used for treating alcoholism and more recently for cocaine addiction. DSF is a relatively nontoxic substance when administered alone, but markedly alters the metabolism of alcohol by irreversibly inhibiting the hepatic aldehyde dehydrogenase (ALDH) and causing an accumulation of acetaldehyde and consequent aversion to further drinking. DSF and its metabolites form mixed disulfide bridges with a critical cysteine (Cys302) near the active site region of ALDH to inactivate the enzyme. Recently, the inventors showed that DSF reacts similarly with a number of redox-sensitive proteins such as the p53 tumor suppressor, NF-κB, and ub-activating enzyme E1 and lead to their degradation. MGMT is highly expressed in about 80% of brain tumors and other cancers. Paradoxically, its antimutagenic function interferes with the cytotoxic actions of anticancer alkylating agents. This is because MGMT effectively repairs the O⁶-methylguanine and O⁶-chloroethylguanine lesions induced by methylating agents (temozolomide, dacarbazine and procarbazine) and chloroethylating agents (BCNU and CCNU) respectively, thereby preventing the generation of mutagenic lesions and interstrand DNA cross-links. Consequently, MGMT has emerged as a central determinant of tumor resistance to alkylating agents. In view of this therapeutic relevance, MGMT has been extensively targeted for inhibitor development. Much success has been achieved through the design of psuedosubstrate inhibitors, namely, the O⁶-benzylguanine (BG) and O⁶-[4-bromothenyl] guanine (Patrin-2), which are currently undergoing clinical trials. In this biochemical strategy, the free base inhibitors (BG) are first administered to inhibit MGMT and create a DNA repair-deficient state followed by alkylating agents to increase the DNA damage and antitumor efficacy. BG is a specific and powerful inhibitor of MGMT and causes a prolonged suppression of DNA repair (48-72 hours) in cultured tumor cells. While this approach has shown a positive outcome in cultured cells and xenograft settings, a significant drawback is the excess of bone marrow toxicity encountered in patients enrolled in BG+alkylating agent combination regimens. Hematopoietic stem cells contain very low levels of MGMT, whose inactivation by BG predisposes them to excessive alkylation damage, which results in therapy discontinuance and necessitates the use of alkylating drugs at sub-therapeutic levels. This problem has prompted a gene therapy approach involving the transduction of BG-resistant MGMT genes (G156A or P140K) in to the hematopoietic stem cells. However, the cost, complexity, and safety issues make this approach cumbersome and impractical. The considerations above justify an urgent need for new and transient inhibitors for human MGMT. To design better and rational inhibitors for MGMT, the reactivity of Cys145 was exploited, which accepts the alkyl residues in the self-inactivating reaction. This active site cysteine has a low pKa of 4.5, and reacts readily with glutathione forming a mixed disulphide linkage. Cysteine 145 of MGMT is also a good substrate for nitrosylation. Therefore, the inventors hypothesized that drugs/small molecules with strong affinity for reactive cysteines will be able bind and inactivate the MGMT protein and disulfiram shows as such a candidate. The present disclosure characterizes MGMT inhibition by DSF, the augmented alkylation damage and a synergistic cytotoxicity and shows an increased carcinogenic risk in alcoholic patients taking disulfiram has also been discussed.

CuDSF was synthesized by mixing equimolar amounts of DSF and CuCl2 for 24 hours followed by extraction with chloroform as described previously. The final product was dried and stored in a desiccator. Hexa-histidine tagged human MGMT was expressed in E. coli and purified as described previously.

The DNA repair activity of MGMT was measured by the transfer of [3H]-labeled methyl groups from the O⁶-position of guanine in the DNA substrate to the MGMT protein as described previously. The DNA substrate enriched in O6-methylguanine was prepared by reacting [3H]-methylnitrosourea (GE Healthcare, 60 Ci/mmol). Briefly, the cell pellets were washed with cold PBS, disrupted by sonication in the assay buffer (30 mM Tris-HCl pH 7.5, 0.5 mM DTT, 0.5 mM EDTA, 5% glycerol, and 20 μM spermidine) and centrifuged. The extracts (50-150 μg protein) were supplemented with the [3H]-DNA (10,000 cpm) and incubated at 37° C. The reactions were terminated by the addition of TCA, the DNA substrate was hydrolyzed at 80° C., and following filtration on glass fiber discs; the radioactivity present in protein precipitates was quantitated.

After trypsinization, the cell pellets were washed with cold PBS, and subjected to sonication in 50 mM Tris-HCl (pH 8.0) containing 1% glycerol, 1 mM EDTA, 0.5 mM PMSF and 2 mM benzamidine and centrifuged. Equal protein amounts from different treatments were electrophoresed on 12% SDS-polyacrylamide gels. Proteins were electro-transferred to Immobilon-P membranes. The membranes were blocked with 5% non-fat dry milk in Trisbuffered saline (TBS; pH 8.0) containing 0.1% Tween 20 for 3 hours, and subsequently incubated with appropriate primary antibodies. Antigen-antibody complexes were visualized by enhanced chemiluminiscence (Pierce Company). Band intensities were quantified using a Bio-Rad Versa Doc Imaging system.

UW228 cells were cultured on sterile coverslips and treated with DSF for 12 hours. The treated and untreated cells were fixed with 4% paraformaldehyde for 20 minutes and washed with PBS. They were blocked with 3% BSA containing 0.2% Triton X-100 for 3 hours. Cells were incubated with the anti-MGMT antibody overnight at 4° C., washed thrice with PBS and incubated with Alexa Fluor-488 goat anti-mouse IgG (Invitrogen) for 1 hour. Cells were counterstained with Hoechst to observe the nuclei, washed and mounted on slides. Images were acquired and quantitated using an Olympus IX 81 fluorescence microscope.

Proteolysis assays in the presence of rabbit reticulocyte lysates (30 μl final vol.) were performed in Tris-HCl (30 mM; pH 8.0) containing 0.5 mM DTT, 4 mM MgCl2, 1 mM ATP, 1 μg ubiquitin, 0.7 μg rMGMT or 0.7 μg DSF treated rMGMT. DSF-exposed MGMT samples were dialyzed to remove the residual drug prior to the degradation assays. The reactions were initiated with the addition of 10 μl rabbit reticulocyte lysate (Promega), incubated at 37° C. for 15-40 minutes, electrophoresed and immunoblotted using anti-MGMT antibodies. The protein bands were quantified by densitometry.

Sixty percent confluent cells were treated with 50 μM DSF for 12 hours. This was followed by 100 μM BCNU. Untreated cells and cells treated with DSF and BCNU alone were used as controls. After the treatments, cells were allowed to grow for 24 and 48 hours. At each of these time points cells were harvested and fixed in 70% ethanol. The cells were then washed with PBS and re-suspended in the presence of RNase (1 μg/ml) and propidium iodide (PI, 50 μg/ml) for 30 minutes. Cell cycle histograms were generated using a BD Accuri C6 flow cytometer.

UW228 cells pre-treated with 50 μM DSF for 12 hours were treated with melphalan (0-48 hours, 100 NM) or BCNU (0-72 hours, 100 NM). DNA from cells was isolated by lysis in TE buffer (10 mM Tris, 1 mM EDTA, pH 8.0) containing 0.5% SDS, and 100 μg/ml RNase A and proteinase K (1 mg/ml) digestion. DNA was precipitated with ethanol and dissolved in TE buffer. The drug-induced DNA crosslinking was measured by the ethidium bromide fluorescence assay as described previously. Briefly, 5-10 μg DNA was dissolved in assay buffer (20 mM potassium phosphate and 2 mM EDTA, pH 11.8) in duplicate. One set of tubes was heated at 100° C. for 10 min and cooled to room temperature. Ethidium bromide was added to 1 μg/ml, and the fluorescence was measured (305 nm excitation and 585 nm emission) using an LS-50 variable wavelength spectrofluorometer (Perkin Elmer). The fluorescence readings were used to compute the crosslink index, CLI, as described earlier.

These were performed using the yellow tetrazolium dye (3-(4, 5-dimethylthiazolyl-2)-2, 5-diphenyl tetrazolium bromide) (MTT) as described previously. Cells (15,000/well) in 24-well plates were treated with DSF (50 μM) for 12 hours before exposure to the alkylating agents. The purple color developed due to formazan was measured at 570 nm using a SPECTRAFluor Plus plate reader (Tecan Inc.). Cell proliferation was also assessed by colony formation assays on soft agar. In these assays, UW228 cells treated with 50 μM DSF were suspended in media containing DMEM, 20% FBS, 0.4% agarose, non-essential amino acids and BCNU (0-75 μM) or TMZ (0-750 μM). Ten thousand cells from these suspensions were layered on 3 ml of the same medium containing 0.6% agar in 60 mm culture plates. Seven to ten days after plating, cell colonies in triplicate plates were counted and cell survival computed.

Mice weighing 19-21 g were administered a single dose of 150 mg/kg DSF dissolved in 50 μl DMSO i.p. injections (6 animals/group). Control mice received DMSO alone. The animals were sacrificed at 1, 3, 6, 9 and 12 hours after drug administration. The liver and brain tissues were harvested, washed, and homogenized in a buffer (0.5% Triton-X, 50 mM Tris-HCl buffer (pH 8.0) containing 3% glycerol, 50 mM NaCl, 1 mM EDTA, 0.5 mM PMSF and 2 mM benzamidine) using a Polytron. The lysates were centrifuged at 12,000 g and the resulting extracts were used for MGMT activity and western blot analyses.

The western blotting and immunocytochemical analyses were performed four times independent of each other. Results were assessed by Student's t test. Significance was defined as P<0.05. Power analysis was used to calculate the number of animals required to achieve a statistical power of >80%.

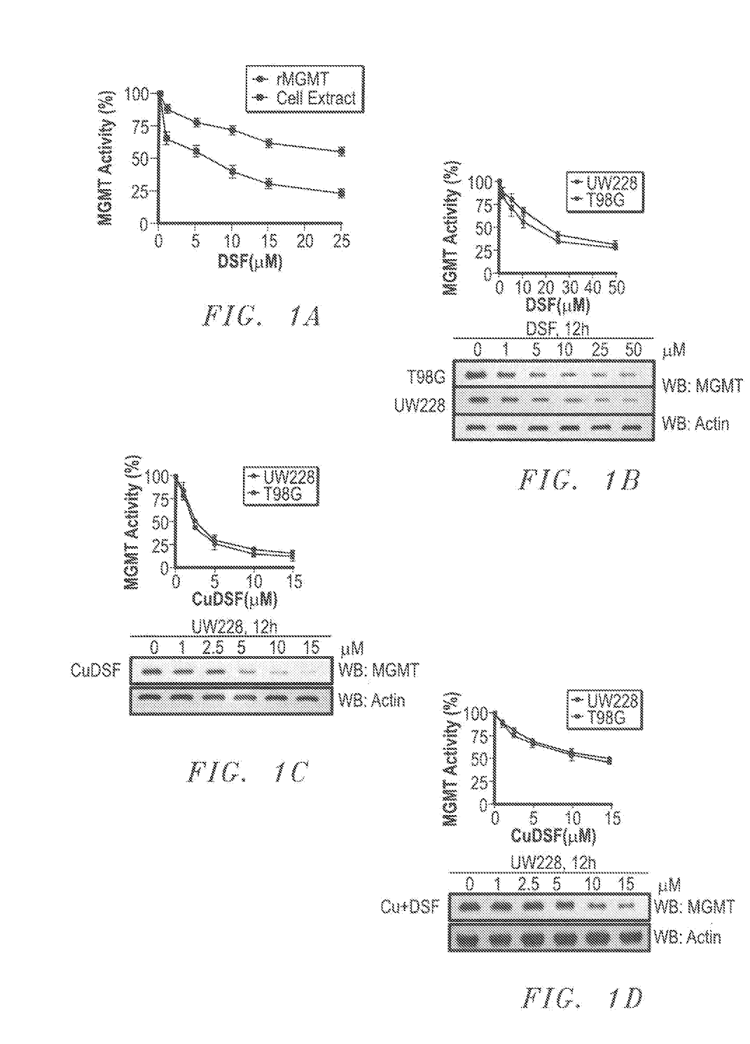

FIGS. 1A-1D show concentration-dependent inhibition of MGMT activity and MGMT degradation induced by DSF and its derivatives in human brain tumor cells. FIG. 1A shows inhibition of the DNA repair activity of purified recombinant MGMT (rMGMT) and cellular MGMT by increasing DSF concentrations. rMGMT or UW228 cell-free extracts were first treated with DSF at concentrations shown for 20 minutes at room temperature followed by the addition of DNA substrate. FIG. 1B shows inhibition of MGMT activity and loss of MGMT protein in T98G and UW228 cells. Tumor cell monolayers were exposed to DSF at concentrations specified for 12 hours. Cells were trypsinized, washed and MGMT activity was determined in cell extracts (top panel). The same extracts were western blotted for MGMT protein. WB, western blot. FIG. 1C shows the effect of CuDSF on MGMT activity and protein levels in UW228 and T98G cells. Cells were exposed to CuDSF and analyzed for MGMT activity and protein as above. FIG. 1D shows the effect of copper and DSF treatments on MGMT. Cells were incubated with 0.1 μM CuCl2 and DSF at concentrations shown for 12 hours before determining the MGMT activity and protein levels. The initial studies involved the treatment of the recombinant MGMT protein (rMGMT) and UW228 cell-free extracts (which contained MGMT) with DSF and quantitation the DNA repair activity. While 25 μM DSF inhibited the purified MGMT by 40%, the repair activity in the extracts was curtailed by 75%, suggesting a direct effect of the compound on MGMT protein (FIG. 1A). Next, the MGMT-proficient human brain tumor cell lines, T98G and UW228 were treated with DSF (0-500 μM) for 12 hours and the MGMT activity was measured in the extracts therefrom. In both cell lines, MGMT activity was inhibited by DSF in a dose-dependent fashion with approx. 70% inhibition in cells exposed to 50 μM DSF. Western blot analysis showed a gradual loss of MGMT protein, consistent with the extent of inhibition after 12 hours DSF-treatment (FIG. 1B). DSF binds copper with a great affinity, and the CuDSF was more potent in this property with 10-15 μM drug eliminating >90% of the MGMT activity and protein in UW228 cells after 12 hours treatment (FIG. 1C). The UW228 cells were exposed to 0.1 μM CuCl2 in combination with DSF; this schedule, however, resulted in modest inhibition of MGMT activity and protein levels (FIG. 1D). These results confirm that DSF and its metabolites are capable of inactivating the MGMT protein and lead to its breakdown in human cancer cells. To delineate the appropriate time for alkylating drug treatments following MGMT inhibition, next, the time course of MGMT depletion by these three interventions was studied.

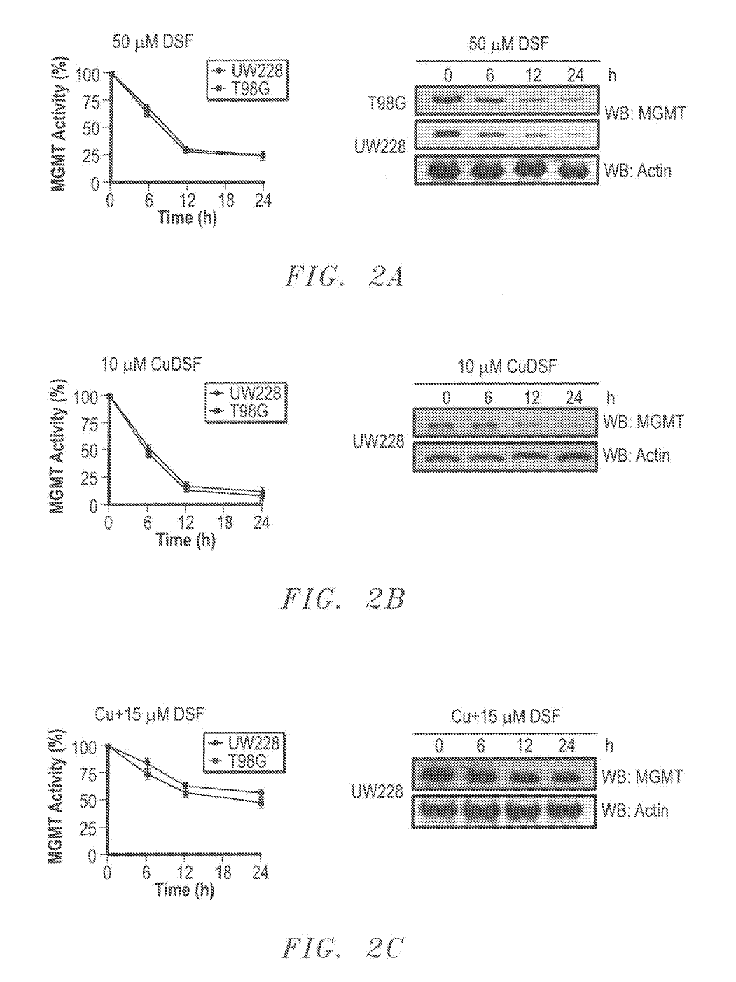

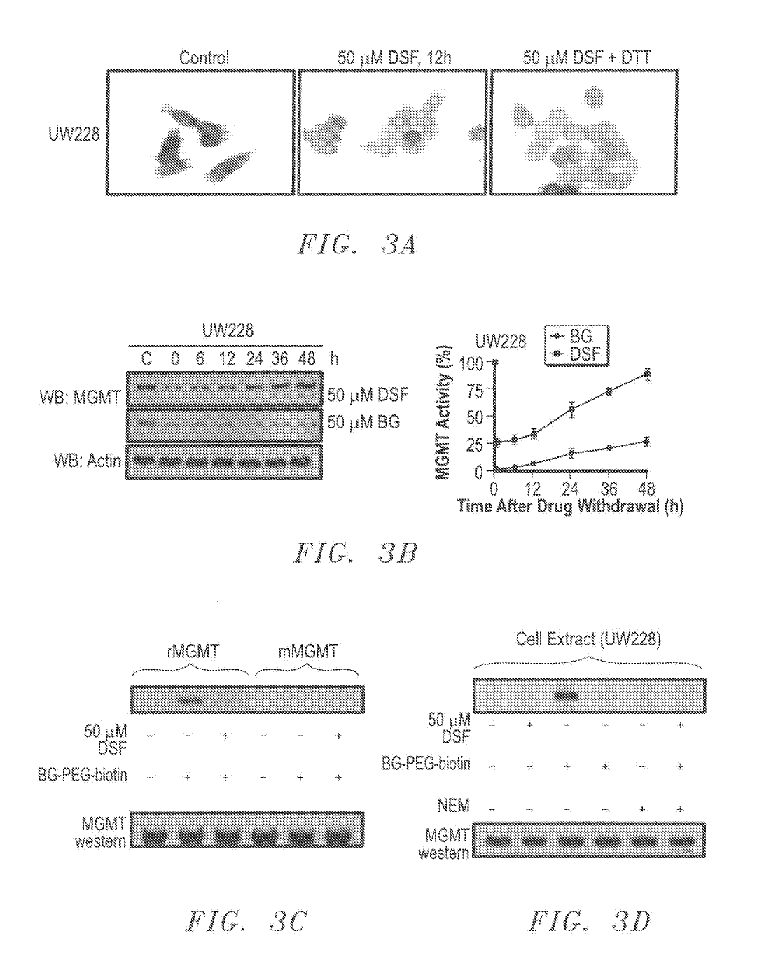

FIGS. 2A-2C show time-dependent elimination of MGMT protein in brain tumor cells by DSF, CuDSF and Cu+DSF. FIG. 2A shows T98G and UW228 cells were treated 50 μM DSF. At times indicated, MGMT activity and protein levels were assessed. FIG. 2B shows potent inactivation of MGMT by 10 μM CuDSF in T98G and UW228 cells. MGMT Protein levels in drug-treated UW228 cells are shown. FIG. 2C shows Inhibition of MGMT in tumor cells after a combined treatment of 0.1 μM CuCl2 and DSF. The presence of 50 μM DSF in the culture medium caused a maximal 75% inhibition of MGMT activity and protein at 12 hours, which was maintained till 24 hours (FIG. 2A). In comparison, CuDSF, at a 5-fold lesser concentration (10 μM) than DSF, elicited a >90% inhibition of MGMT activity and protein during the same period (FIG. 2B). Consistent with the results from FIG. 1D, the combination of Cu and DSF was less potent, with only 40-50% inhibition (FIG. 2C). Collectively, the findings in FIGS. 1 and 2 reveal that DSF is capable of rapidly inactivating the MGMT and maintain the inhibition to allow the induction of increased alkylation damage.

FIG. 3A shows immunoflourecence decreased MGMT protein levels in DSF-treated UW228 cells. Drug treatments and exposure times are shown in labels. In the last panel, DSF-treated cells were exposed to 10 mM DTT for 20 min before the antibody treatment. FIG. 3B shows depletion and repletion kinetics of MGMT protein following BG and DSF treatments. UW228 cells were exposed to 50 μM DSF or 50 μM BG for 12 hours followed by washing and suspension of cells in drug-free media. Cells were trypsinized at times shown. Levels of MGMT activity and protein were determined in cell extracts. Evidence that DSF blocks the active site cysteine (cys145) in human MGMT protein. FIG. 3C shows evidence that Cys145 of MGMT is the target site for DSF. Wild type rMGMT and mutant rMGMT (C145A) proteins were incubated with 50 μM DSF in 40 mM Tris-HCl, pH 8.0, 1 mM EDTA for 20 minutes at 37° C. BG-PEG-biotin (5 μM) was then added and incubations continued for 15 minutes. The samples were electrophoresed, blotted and probed with Streptavidin-HRP to detect the protein-bound biotin (upper panel). The blot was reprobed with antibodies to MGMT (lower panel). FIG. 3D shows DSF abrogates the binding of the BG probe to cellular MGMT. UW228 cell extracts proficient in MGMT were incubated with DSF or N-ethylmaleimide (0.5 mM) for 20 minutes followed by BG-PEG-biotin as described for the purified proteins. Streptavidin-HRP was used to probe the resulting blot (upper panel). The blot reprobed with MGMT antibody shows equal protein loading (lower panel).

To confirm the data obtained by western blotting in FIGS. 1A-D and 2A-C, immunostaining of MGMT in UW228 cells treated with DSF was performed. The representative photomicrographs in FIG. 3A shows that the antibody-specific staining for MGMT was markedly less in DSF-treated cells. To test whether the thiol linkages introduced by DSF on the protein are reversible, in some experiments, the DSF-treated cells were exposed to 10 mM DTT for 30 minutes prior to treatment with MGMT antibodies. The intensity of antibody staining did not change after DTT, indicating the non-reversibility of the modification. Further, DTT was unable to reverse the inhibition of MGMT activity by DSF (not shown). Thus, unlike the mixed disulfides introduced in S-thiolation reactions, the disulfiram linkages introduced in the MGMT protein appear largely irreversible.

MGMT performs a stoichiometric reaction to accomplish the DNA repair and is not recycled to its active form. Instead, the inactivated protein is degraded and fresh translation has to occur to restore the MGMT levels in cells. The rate and extent of this recovery is an important factor in the extent of damage introduced to the normal cell genomes such as the bone marrow stem cells. Therefore, the MGMT depletion and repletion kinetics after DSF treatment was compared with that by BG, an established inhibitor of MGMT, currently in clinical trials. In these experiments, the UW228 cells were incubated with 50 μM DSF or 50 μM BG for 12 hours, and then, the cultures were washed to remove the residual drugs followed by resuspension in drug-free media to allow MGMT regeneration. MGMT protein and activity levels were analyzed during the depletion and repletion (FIG. 3B). While BG resulted in 100% loss of MGMT activity, DSF produced a 75% inhibition at 12 hours post-drug treatments (FIG. 3B, right panel). However, the post-recovery of MGMT activity was very slow in BG-treated cultures compared to that in DSF treatment. While the MGMT activity recovered to 25% of control levels at 48 hours post-BG, it reached ˜95% in case of post-DSF at the same interval (FIG. 3B, right panel). MGMT protein levels as determined by western analyses during the depletion/repletion cycle were consistent with and fully reflected the DNA repair activity profiles (FIG. 3B, left panel). These data suggest that DSF functions as a transient inhibitor of MGMT, and that DSF inactivated MGMT may undergo an accelerated proteolysis compared to the benzylated MGMT protein. Since the longer MGMT suppression is likely to mediate a continued accumulation of alkylation DNA damage, our observations suggest that in contrast to BG, the DSF-induced short term inhibition of MGMT is likely to be beneficial in rescuing the host tissues from continued genomic injury. The human MGMT protein possesses 5 cysteine residues, of which the Cys145, which accepts the alkyl groups, is the most reactive. In the first approach, to test whether DSF reacts with cys145, a biotin-labeled BG probe was used, which binds specifically to this cysteine residue. Purified rMGMT protein was incubated with BG-PEG-biotin before or after treatments with DSF, followed by SDS-PAGE and detection of the protein-bound biotin using the streptavidin-HRP on the blots. When the protein was exposed to DSF followed by BG-PEGbiotin, there was a significant reduction in binding of the probe (FIG. 3C, lanes 1-3). Further, the purified mutant MGMT protein was used in which the Cys145 has been replaced with alanine in this assay. The BG-biotin probe failed to bind this mutant protein with or without DSF treatment (FIG. 3C, lanes 4-6). When the entire blot was reprobed with MGMT antibody, the rMGMT used in the assays was detected at equal levels (FIG. 3C, lower panel). Similarly, when the MGMT containing cell extracts were treated or untreated with DSF prior to labeling with the BG-PEG-biotin probe, DSF eliminated the western blot signal (FIG. 3D). Nethylmalamide (NEM) used as a control to block the active site cys145 of MGMT, also prevented the BG-labeling of the protein (FIG. 3D). These data clearly demonstrate that DSF modifies the active site cys145 of MGMT, which in turn, inactivates the DNA repair function.

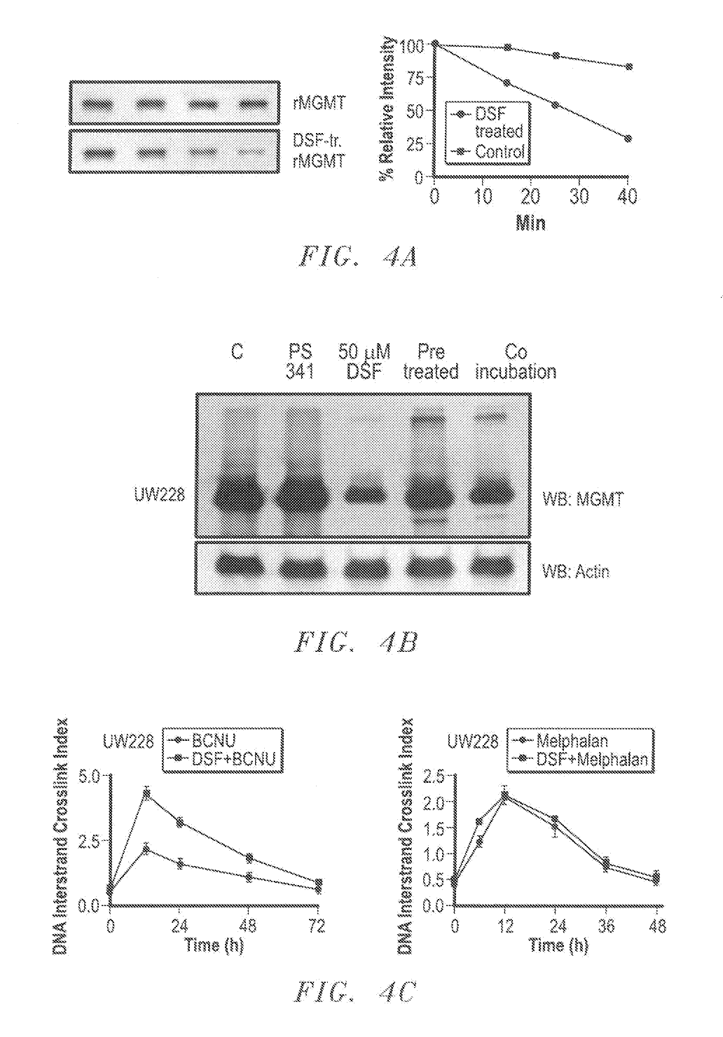

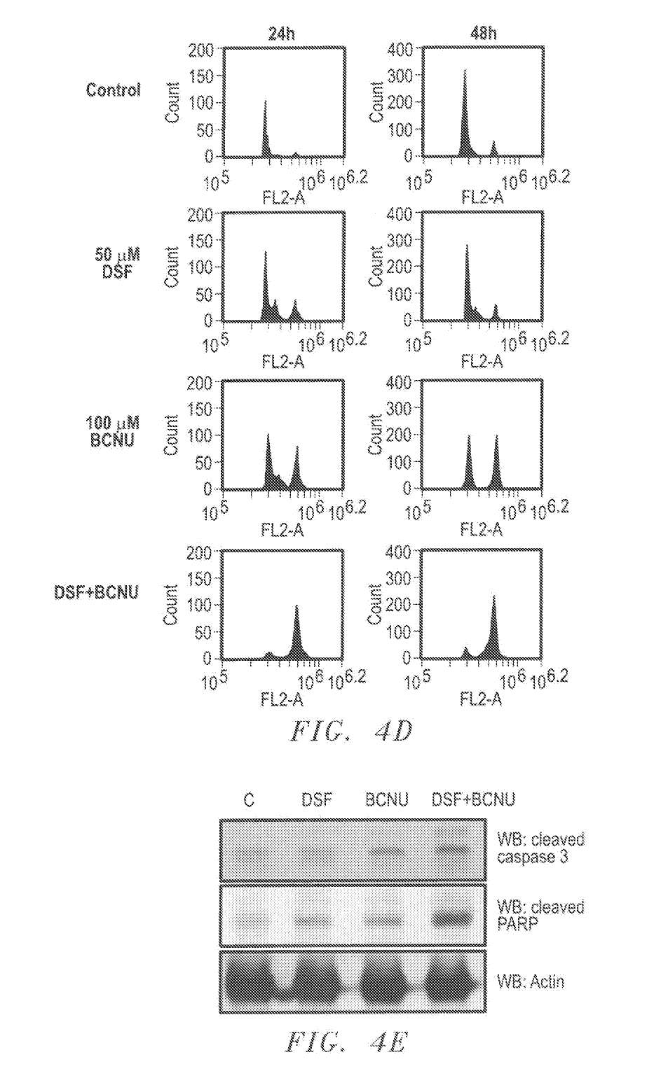

FIGS. 4A-4E show proteasomal degradation of DSF modified MGMT. FIG. 4A shows In vitro degradation of DSF treated rMGMT protein. 0.5 μg Unmodified rMGMT and DSF treated rMGMT (after dialysis) proteins were subjected to ATP and Mg2+-dependent degradation in rabbit reticulocyte lysates for 0-40 min as described in Methods. The samples were immunoblotted and probed with MGMT antibodies (right panel). The densitometric quantitation of protein bands on the western blots is shown as a line graph on the left. FIG. 4B shows the proteasome inhibitor PS-341curtails the degradation of DSF-modified MGMT protein in brain tumor cells. UW228 cells were treated with 10 μM PS341 alone for 6 hours (lane 2), with 50 μM DSF alone for 12 hours (lane 3), pretreated 6 hours with 10 μM PS341 followed by 50 μM DSF for 12 hours (lane 4) and co-incubated with PS341 and DSF for 6 hours (lane 5). Cell lysates were western blotted for the MGMT protein. FIG. 4C shows DSF increases the DNA damage induced by MGMT-targeted alkylating agents. Kinetics of DNA interstrand crosslinks formed in UW228 cells after treatment with 100 NM BCNU or 100 NM melphalan is shown. Melphalan does not produce O6-alkylguanines, and served as a control. Cells were treated or untreated with 50 μM DSF for 12 hours to deplete the MGMT protein. They were then exposed to BCNU or melphalan. At times specified, the cells were harvested, DNA isolated and the extent of interstrand crosslinking of DNA was determined using the ethidium bromide fluorescence assay. Values are mean+S.D. The results were significant at P<0.05. FIG. 4D shows the effect G2/M blockade induced by BCNU is enhanced and extended in the presence of DSF. Histograms showing results of FACS-based cell cycle analysis of UW228 cells treated with BCNU or DSF+BCNU. Unsynchronous cultures were treated or untreated with DSF (50 μM for 12 hours) and then incubated with 100 μM BCNU. The cells were trypsinized at times indicated and analyzed by flow cytometry. FIG. 4E shows Enhanced levels of apoptosis markers—cleaved caspase 3 and cleaved PARP after DSF+BCNU treatments compared with BCNU alone.

Since the MGMT inactivated by BG is degraded through ub-proteolysis, it was of interest to investigate the mode of elimination of DSF-modified MGMT protein. Therefore, first, we compared the in vitro degradation of DSF-treated and untreated rMGMT protein in rabbit reticulocyte lysates (RL), which are known to promote the proteasomal degradation. The control and DSF-modified MGMT proteins were incubated with RL in the presence of Mg++, ATP and ubiquitin at 37° C. The reaction mixtures were blotted and probed with MGMT antibodies. The resulting western blot and the densitometric quantitation of protein bands are shown in FIG. 4A. The data shows that the untreated rMGMT remained largely undegraded during the 40 minutes incubation, whereas the DSF-modified protein disappeared gradually, 30% at 10 minutes, 45% at 25 minutes and 70% at 40 minutes of incubation (FIG. 4A). To show the involvement of the ub-proteolysis in cells a clinically used proteasome inhibitor. UW228 cells were treated with PS-341 alone for 6 hours, DSF alone, exposed to DSF after PS341, or co-incubated with PS341 and DSF, and the lysates were processed for western blotting of MGMT. PS341 co-incubation with DSF decreased the MGMT degradation by just 20%, whereas the cells pre-exposed to PS341 and then to DSF, showed a >50% reduction in the loss of MGMT protein (FIG. 4B, lanes 4-5). Taken together, the results presented in FIGS. 4A-4B confirm that DSF-modified MGMT is perceived as a structurally altered/damaged protein in cells and eliminated through the ub-proteasomal route.

As discussed in the Introduction, the inhibition of MGMT-mediated DNA repair is expected to augment the levels of alkylation DNA damage and the interstrand crosslinking of DNA induced by bifunctional alkylating agents. This hypothesis was tested by treating the UW228 cells with DSF for 12 hours followed by BCNU or melphalan. An ethidium bromide fluorescence assay was used to determine the levels of cellular DNA crosslinking. Cells treated with DSF and BCNU showed an approximately 2-fold increase in interstrand crosslinks as compared to BCNU alone (FIG. 4C). However, DSF was unable to increase the crosslinks generated by melphalan. While BCNU introduces the chloethyl groups to the O⁶-position of guanine, which are substrates for MGMT, melphalan is not an O⁶-guanine alkylator, but predominantly generates N7-guanine alkylations; thus, MGMT does not interfere with the damage induced by melphalan, and this explains the inability of DSF to enhance the melphalan-induced DNA crosslinking.

To determine the consequences of MGMT inactivation by DSF on cell cycle changes induced by BCNU, we performed flow cytometry (FIG. 4D). UW228 cells were treated with DSF alone, BCNU alone and a combination of DSF and BCNU, and histograms were generated from propidium iodide-stained cells. The results indicate that 50 μM DSF alone did not induce any cell cycle changes after 48 hours. Treatment with BCNU, as expected, produced a significant G2/M blockade (33% of cells) at 24 hours. Further, the cells pretreated with DSF and then exposed to BCNU showed a very strong accumulation of cells (˜80%) in G2/M phase at 24 hours, and this blockade was maintained at the same level at 48 hours (FIG. 4D). In DSF-treated UW228 cells, the greater level of DNA damage induced by BCNU (FIG. 4C) and the resulting lengthy cell cycle arrest (FIG. 4D) were associated with the activation of apoptotic machinery as reflected by an increased expression of cleaved caspase 3 and cleaved PARP proteins (FIG. 4E). Collectively, the evidence provided so far, distinctly indicates that the nontoxic drug DSF, acting through the inhibition of MGMT, is highly capable of potentiating the cytotoxic effects of alkylating agents.

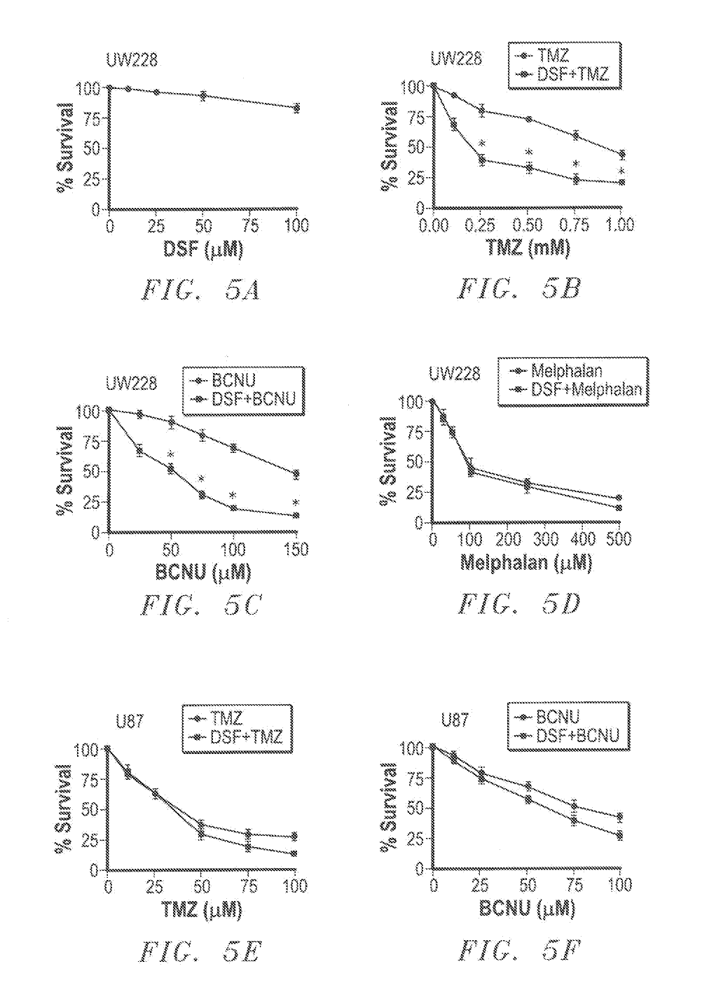

FIGS. 5A-5G show DSF preexposure sensitizes the brain tumor cells to the clinically used alkylating agents. FIG. 5A shows cell survival after DSF treatment alone. UW228 cells were treated with increasing concentrations of DSF (1-100 μM) for 24 hours. Cells were then cultured for 48 hours before performing the MTT assays. Since a 50 μM concentration for DSF generated a less than 5% cell killing, this concentration was chosen to potentiate the alkylating drugs. FIG. 5B shows TMZ mediated cell killing with and without DSF preexposure in UW228 cells. FIG. 5C shows BCNU mediated cell killing with and without DSF preexposure in UW228 cells. FIG. 5D shows DSF did not increase the cytotoxicity of melphalan, an N7, but not an O⁶-alkylator of guanine in UW228 cells. FIG. 5E shows DSF did not potentiate the TMZ induced cytotoxicity in the MGMT-deficient U87 malignant glioma cells. FIG. 5F shows DSF did not potentiate BCNU-induced cell killing in the MGMT-deficient U87 cells. In all these cases, the tumor cells were treated or untreated with 50 μM DSF for 12 hours followed by the alkylating drugs for 4 days before the MTT assays. FIG. 5G shows soft agar colony formation assay for cell survival analysis. UW228 cells were treated or untreated with 50 μM DSF prior to BCNU or TMZ exposures as described in Methods. The effect of drugs on cell survival was computed at each concentration. The data represents the results of three independent experiments performed in triplicate. Values are mean+S.D. The results presented in panels B, C and G (at 25-75 μM BCNU and 250-750 μM temozolomide) were significant at P<0.05. *, indicates statistically significant difference as compared to controls.

DSF can increase the efficacy of many anticancer drugs. In the context of this study, we performed cell survival assays using the MTT to determine the impact of DSF on alkylator-mediated tumor cell killing. DSF by itself, over a concentration range of 10-100 μM for 24 hours showed no toxic effects on UW228 (FIG. 5A) and other tumor cells (not shown). Based on this, we choose 50 μM DSF preincubation for 12 hours to test the potentiation of alkylating drugs. In this setting, TMZ (0-750 μM)+DSF combination showed a 4 to 5-fold increased cytotoxicity compared to TMZ alone (FIG. 5B). For BCNU, the potentiation was 3-fold (FIG. 5C). To confirm the MGMT-specific effects of DSF in cell survival assays, we used two types of controls. First, the drug melphalan, which does not generate O⁶-alkylguanines was used and DSF did not increase the melphalan-induced cell killing (FIG. 5D). Second, the effects of TMZ and BCNU were tested in DSF-pretreated U87 malignant glioma cells, which lack MGMT expression due to promoter methylation. DSF did not increase the extent of cell killing by TMZ or BCNU in U87 cells (FIGS. 5E-F).

Colony formation assays on soft agar were also carried out to confirm the DSF-induced increases in tumor cell killing. DSF treatment of UW228 cells followed by BCNU or TMZ exposures resulted in a 3-fold enhanced cytotoxicity for both drugs (FIG. 5G). The cell survival assays, again, demonstrate that MGMT inhibition by DSF is a significant factor in amplifying the cytotoxic effects of the clinically used alkylating agents.

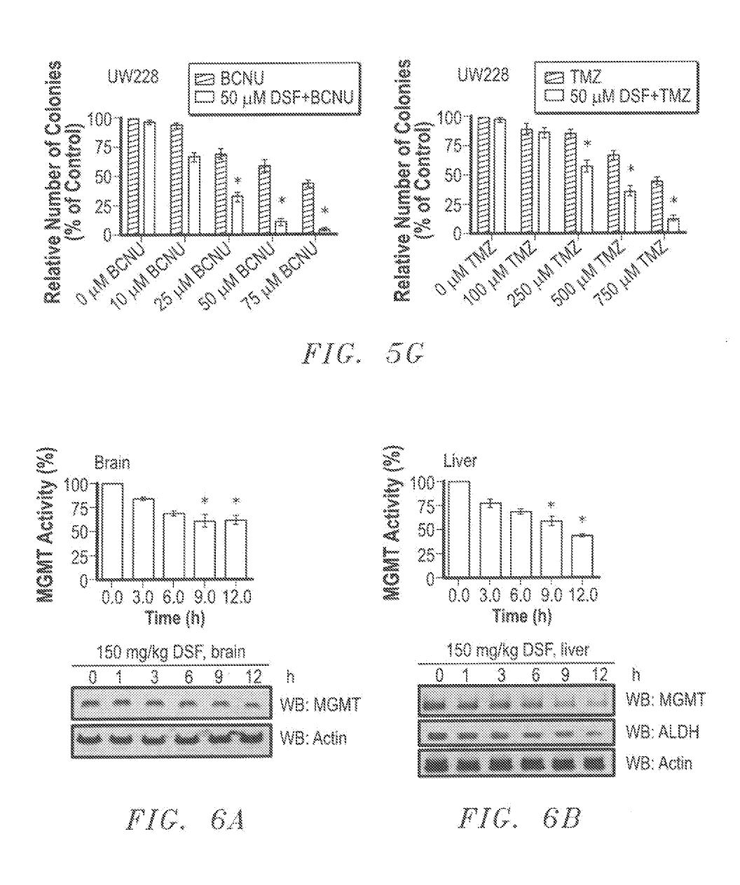

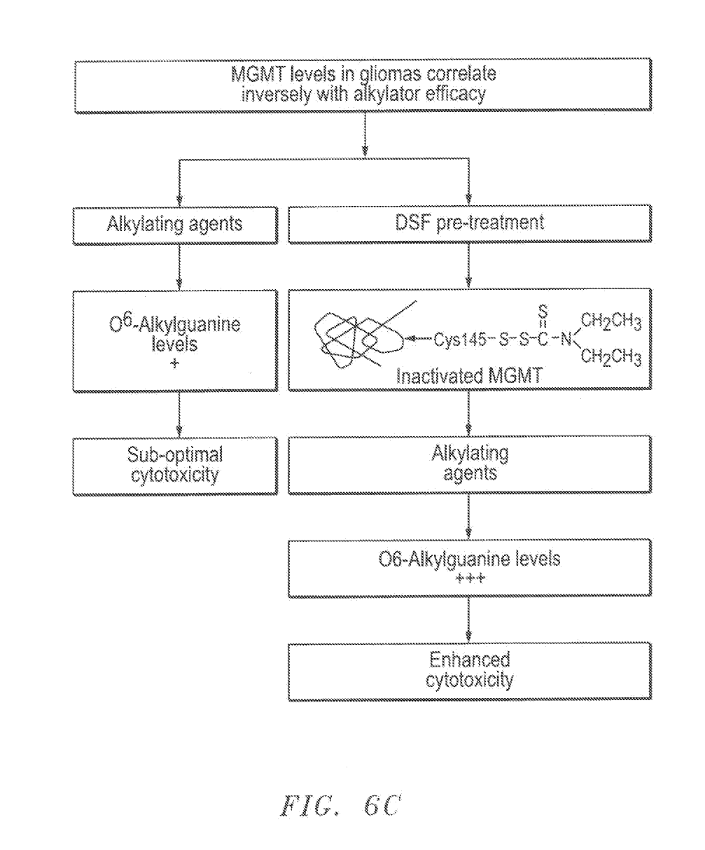

FIGS. 6A-6C show significant downregulation of MGMT activity and protein in mouse brain and liver tissues after DSF administration. Animals in groups of six were given i.p. injections of a single DSF dose (150 mg/kg). The mice were sacrificed at 3, 6, 9 and 12 hours post-DSF. Liver and brain tissue lysates were prepared as described in Methods and used for MGMT activity assays and western blotting. FIG. 6A shows DSF treatment resulted in a sustained 40% inhibition of brain MGMT activity starting at 9 hours (upper panel). Western blot analysis (lower panel) shows a gradual decrease of MGMT protein in the same time course. FIG. 6B shows decreased hepatic MGMT activity (60% inhibition, upper panel) and protein levels (lower panel) after DSF administration. The decrease of MGMT activity in DSF-treated animals in both liver and brain were significant as compared to the values in mice treated with the vehicle alone. *, P<0.05 compared with controls. Error bars indicate SD. Western blot analysis was also performed for ALDH (a major target of DSF) in liver lysates, and decreased protein levels are evident. FIG. 6C is a scheme showing the consequences of MGMT inhibition by DSF and increased in tumor sensitivity to alkylating agents. The overall findings of the present study are summarized. The active site cysteine (Cys145) of MGMT which accepts the alkyl groups in a self-inactivating reaction is highly reactive. The electrophilic DSF and its metabolites can form adducts with this cysteine and inactivate the MGMT-catalyzed removal of alkylation DNA damage. Subsequent administrations of alkylating agents induce higher levels of the cytotoxic O6-alkyguanine lesions.

To further illustrate MGMT inhibition by DSF in a preclinical setting, a single dose of DSF (150 mg/kg, i.p.) was administered and the liver and brain tissues isolated 1, 3, 6, 9 and 12 hours post-injection, and determined MGMT activity in clarified tissue lysates. MGMT activity in 6 animals at each time point was averaged and used to compute the changes in DNA repair activity. There was a gradual reduction of MGMT in brain with a 40% decrease of the repair activity occurring at 9-12 hours (FIG. 6A, upper panel). The MGMT protein levels correlated well with the observed changes in the activity (FIG. 6A, lower panel). In liver tissues, MGMT inhibition was much stronger with a 60% decrease observed at 12 hours relative to the controls (FIG. 6B, upper panel). Hepatic MGMT protein appeared to undergo degradation in DSF treated animals, in a manner similar to that observed in cancer cell lines (FIG. 6B, lower panel). Significantly, the hepatic levels of ALDH protein, the primary target of DSF mediating the alcohol antagonism, also showed a progressive decline in DSF-treated animals, much similar to that observed for the MGMT protein. To our knowledge, this is the first report showing a decrease of ALDH protein in DSF-treated animals. Overall, the data obtained in animal studies establish that DSF can curtail the MGMT activity in the brain and strengthens the drug's utility in brain tumor therapy.

Recently, as a part of an international effort, we proposed that a cocktail of 9 pharmacologically well-characterized nontoxic drugs, collectively called CUSPS be added to a continuous low dose temozolomide for improving survival and quality of life for relapsed glioblastoma patients, who have one of the worst 5-year survival rates among all human cancers. The nine adjuvant drug regimen consists of the aprepitant, artesunate, auranofin, captopril, copper gluconate, disulfiram, ketoconazole, nelfinavir, and sertraline. Of these drugs, disulfiram has the strongest evidence of potential benefit in CNS cancers. The copper gluconate in the regimen is meant to enhance the efficacy of DSF, because the copper bound drug has been shown to be more potent and exert higher levels of cytotoxicity. In fact, the ongoing clinical trials of DSF in metastatic cancers of the liver, prostate, and melanoma include the copper gluconate or zinc gluconate as a component. Consistent with this premise, we found that copper-chelated DSF was about 5-fold more potent than DSF in inhibiting the MGMT activity in cultured brain tumor cells (FIGS. 1B, 1C).

One embodiment of the present invention is summarized in FIG. 6C. Chemically, DSF has a symmetrical structure and its first metabolic step is the reduction of the disulfide group at the center of the molecule to yield two diethyldithiocarbamate (DDC) moieties. DDC is further converted to its methyl ester, and other metabolites. DDC is a potent inhibitor of ALDH, forming mixed disulfide bridges with a critical cysteine near the active site. Our data from the cell culture and animal studies indicate that DSF interacts with Cys145 of MGMT the same way to inactivate the DNA repair protein (FIG. 6C). The covalent adducts induced by DSF alter the secondary and higher order structure of the MGMT protein, allowing the inactivated protein to be recognized by the ubiquitin conjugating enzymes. Evidence clearly pointing to the involvement of the ubiquitination-dependent proteolysis in the processing of DSF-conjugated MGMT was obtained (FIG. 4A, B). Our recent findings that DSF mediates the degradation of several redox-sensitive proteins (p53, NF-κB, ub-activating enzyme E1) in tumor cells clearly agree with the observations made here. The covalent adduct introduced by DSF is non-reducible at physiological concentrations of GSH, and actually, the endogenous GSH has been reported to increase the inactivation of ALDH by DSF metabolites. Similarly, it was found that MGMT inactivation by DSF was not reversible by thiols (DTT or GSH).

One surprising and unexpected result was that DSF has MGMT-dependent biochemical effects. Also clear from the literature is that DSF has multiple cellular targets and promotes the efficacy of anticancer agents of different classes in a pleiotropic “MGMT independent” manner as well. We showed potent inhibition of MGMT activity and the resulting protein degradation in tumor cell lines and animal tissues. BG, the MGMT inhibitor in clinical trials, has also been shown to inhibit MGMT in normal tissues in animals and humans. DSF, in contrast to BG, appeared to be a weaker inhibitor of MGMT causing a short-term inactivation of MGMT >30 hours, which is still sufficient for enhancing the efficacy of alkylating agents, because, the alkylation of DNA is a rapid process. The promotion of BCNU-induced DNA crosslinking in DSF treated cells (FIG. 4C) again supports this postulate. Moreover, the faster regeneration of MGMT following DSF treatment is likely to decrease the toxicity to the normal tissues such as the marrow.

From the discussion above, it is clear that DSF has a huge potential for therapy of CNS tumors. It is a drug already approved by regulatory agencies for human use, its pharmacokinetics and safety issues are well known; this should enable a faster clinical application. DSF has a favorable lipophilic profile (Molecular weight 296.5 Daltons, partition coefficient Log P of 2.8) to cross the blood-brain barrier, and many studies have established its entry in to the brain. Further, disulfiram administered in the absence of alcohol is largely non-toxic and tolerated very well. Although no precise data is available in humans, DSF can be given up to 1 g/day in adjuvant settings to non-alcoholic patients. In rats, DSF at high doses of 600 mg/kg daily for 3 days or smaller doses (up to 100 mg/kg) at twice a week dosing has been shown not to exert any liver toxicity. Also noteworthy is that DSF appears to be selectively toxic to human cancer cells compared to the normal cell counterparts; thus, in pairs of the chronic lymphocytic leukemia and normal lymphocytes, invasive cancer and normal endothelial cells, the glioblastoma and normal astrocytes, there was a preferential killing of cancer cells by DSF. The redox-sensitive nature of the human MGMT protein was exploited and since DSF is (i) nontoxic, (ii) is hydrophobic enough to cross the blood-brain barrier, (iii) unlikely to induce tumor resistance, (iv) has MGMT-independent signaling effects that may actually promote the chemotherapy, the ability of DSF to create an MGMT-deficient state provides for improving the brain tumor therapy.

In addition the present disclosure shows that chronic alcoholics undergoing disulfiram therapy for a long time are likely to have an increased risk for developing cancer. This is because, alcohol, by itself is known to downregulate the MGMT activity. DSF, through a direct effect reported here, can further exacerbate the MGMT inactivation, and the ability of different organs to defend themselves against the endogenous and environmentally derived alkylating agents may be compromised. Such an unrepaired DNA damage, particularly in the regulatory oncogenes and tumor suppressor genes, may manifest in harmful mutations and promote the genomic instability. The reported occurrence of frequent mutations in K-ras, p53 and β-catenin genes in cells with reduced MGMT activity or silencing of the MGMT gene through promoter methylation in human cancers is consistent with this premise.

NCX-4016 (NO-Aspirin), 2-(acetyloxy) benzoic acid 3-[(nitrooxy) methyl]phenyl ester, is an acetylsalicylic acid molecule linked at the meta position to a chemical spacer (hydroxybenzylalcohol) bearing the NO-donating moiety (benzenemethanol-3-hydroxy-á-nitrate or NCX 4015). It is metabolized in the liver and plasma by esterases to yield the parent drug and a (nitromethoxy) phenol which is the spacer attached to the NO releasing moeity. Further biotransformation of the (nitromethoxy) phenol yields NO-3 at a much slower rate. Reactive (ionized) cysteines present on protein surfaces perform essential functions and are preferentially attacked by ROS and RNS. The consequences of cysteine oxidation and protection against these oxidations by reversible and dynamic glutathionylation and nitrosylation, and their interrelationships are shown. MGMT is overexpressed in human cancers and thereby decreases the formation of mutagenic O6-alkylguanine adducts and cytotoxic DNA interstrand crosslinks. Currently, O6-benzylguanine (BG), a potent inhibitor of MGMT is in clinical trials for increasing the efficacy of alkylating agents. However this strategy is beset with the development of BG resistance, and cumulative myelosuppression. The compounds were tested which posttranslationally modify the active site Cys145 (a reactive cysteine) as a new strategy for powerful inhibition of MGMT.

Nitrosylation or covalent modification of reactive cysteines present in many cancer chemotherapy target proteins was designed as a new anticancer strategy. Two non-toxic drugs, namely, disulfiram (a thiolating agent) and nitroaspirin (nitrosylating agent) were used in our approach. Most significantly, nitroaspirin caused a rapid inhibition and degradation of the DNA repair protein MGMT at physiologically achievable concentrations. The extent of inhibition and elimination of MGMT protein by nitroaspirin was well-comparable with those achieved by O6-benzylguanine (BG), an MGMT inhibitor currently undergoing clinical trials. Therefore, the modulation of the active-site Cysteine 145 (pKa=6.4) of MGMT by redox-modifying compounds will provide new and superior therapeutic avenues for enhancing the antitumor efficacy of clinically-used alkylating agents, without the harmful bone-marrow suppressive effects seen with the BG+alkylator strategy. A dramatic and dose-dependent degradation of many redox-regulated proteins (p53, GST-π, NFκB, MGMT, and p21cip1) by disulfiram was also observed.

Numerous non-cancer diseases involve excessive or hyperproliferative cell growth, termed hyperplasia. As used herein, the terms “proliferative disorder”, “hyperproliferative disorder,” and “cell proliferation disorder” are used interchangeably to mean a disease or medical condition involving pathological growth of cells. Such disorders include cancer.

Non-cancerous proliferative disorders include smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis, desmoid tumors and the like.

Suitable pharmaceutically acceptable carriers may contain inert ingredients which do not inhibit the biological activity of the disclosed disalts. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, Mack Publishing Company, Easton, Pa. Formulation of the compound to be administered will vary according to the route of administration selected (e.g., solution, emulsion, capsule). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrins) are known in the art (Baker, et al., “Controlled Release of Biological. Active Agents”, John Wiley and Sons, 1986).

The compounds of the invention are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds of the invention can also be administered orally (e.g., dietary), topically, by inhalation (e.g., intrabronchial, intranasal, oral inhalation or intranasal drops), or rectally, depending on the type of cancer to be treated. Oral and parenteral administrations are preferred modes of administration.

As used in accordance with this invention, the term providing an effective amount means either directly administering such a compound of this invention, or administering a prodrug, derivative, or analog which will form an effective amount of the compound of this invention within the body.

Many new drugs are now available to be used by oncologists in treating patients with cancer. Often, tumors are more responsive to treatment when anti-cancer drugs are administered in combination to the patient than when the same drugs are administered individually and sequentially. One advantage of this approach is that the anti-cancer agents often act synergistically because the tumors cells are attacked simultaneously with agents having multiple modes of action. Thus, it is often possible to achieve more rapid reductions in tumor size by administering these drugs in combination. Another advantage of combination chemotherapy is that tumors are more likely to be eradicated completely and are less likely to develop resistance to the anti-cancer drugs being used to treat the patient.

Optionally, a compound of the invention, or a tautomer, pharmaceutically acceptable salt, solvate, clathrate, or a prodrug thereof, can be co-administered to treat a patient with a proliferative disorder such as cancer, or to prevent the reoccurrence of a proliferative disorder such as cancer, with other anti-cancer agents such as Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazornycin; edatrexate; eflornithine hydrochloride; elsamitrucin; enloplatiiLeniiromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; viriorelbine tartrate; vinrosidine sulfate; vinzolidine sulfate; vorozole; zeniplatin; zinostatin; zorubicin hydrochloride.

Other drugs that can be used in combination with the compounds of the invention to treat a patient with a proliferative disorder such as cancer, or to prevent the reoccurrence of a proliferative disorder such as cancer, include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithineaelemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O⁶-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2, proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; rarnosetran; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

It is understood that the effective dosage of the active compounds of this invention may vary depending upon the particular compound utilized, the mode of administration, the condition, and severity thereof, of the condition being treated, as well as the various physical factors related to the individual being treated. As used in accordance with invention, satisfactory results may be obtained when the compounds of this invention are administered to the individual in need at a daily dosage of from about 0.001 mg to about 100 mg per kilogram of body weight, preferably administered in divided doses two to six times per day, or in a sustained release form. For most large mammals, the total daily dosage is from about 1.5 mg to about 1000 mg. It is preferred that the administration of one or more of the compounds herein begin at a low dose and be increased until the desired effects are achieved.

Such doses may be administered in any manner useful in directing the active compounds herein to the recipient's bloodstream, including orally, via implants, and parenterally (including intravenous, intraperitoneal and subcutaneous injections). Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations herein may utilize standard delay or time release formulations to alter the absorption of the active compound(s).

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A pharmaceutical composition comprising an effective amount of a disulfiram composition in a pharmaceutically acceptable carrier for use in the treatment of tumor cells wherein the disulfiram composition inhibits an O⁶-methylguanine DNA methyltransferase in the tumor cells.

2. The pharmaceutical composition according to claim 1 wherein disulfiram composition comprises disulfiram, copper-chelated disulfiram, copper-chloride disulfiram or a combination thereof.

3. The pharmaceutical composition according to claim 1 wherein the disulfiram composition is administered in combination with a metal chelate that includes an ion selected from the group consisting of arsenic, bismuth, cobalt, copper chromium, gallium, gold iron, manganese, nickel, silver, titanium, vanadium, selenium and zinc.

4. A pharmaceutical composition comprising an effective amount of a nitrosylating agent in a pharmaceutically acceptable carrier to inhibit O⁶-methylguanine DNA methyltransferase in tumor cells.

5. The pharmaceutical composition according to claim 4 wherein the nitrosylating agent is nitroaspirin.

6-8. (canceled)

9. The pharmaceutical composition according to claim 1 wherein the tumor cells are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

10. The pharmaceutical composition according to claim 1 further comprising a cytotoxic agent selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus, deforolimus, paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydroxy-3,5-dimethoxyphenyl]-5,5a,6,8,8a,-9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-py-razolo[4,5,1-de]acridin-6-one, N-[1-[2(diethylamino)ethylamino]-7-methoxy-9-oxo-9H-thioxanthen-4-ylmethy-1]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna.

11-12. (canceled)

13. A pharmaceutical composition for inhibiting DNA repair in subject suffering from a proliferation disorder, wherein the pharmaceutical composition comprises an effective amount of a cytotoxic agents and a disulfiram composition, a nitrosylating agent or both in a pharmaceutically acceptable carrier to inhibit an O⁶-methylguanine DNA methyltransferase.

14. The composition according to claim 13 wherein disulfiram composition comprises disulfiram, copper-chelated disulfiram, copper-chloride disulfiram or a combination thereof.

15. The composition according to claim 13 wherein the nitrosylating agent is nitroaspirin.

16. The composition according to claim 13 wherein the nitrosylating agent is nitroaspirin and the disulfiram composition is disulfiram, copper-chelated disulfiram, copper-chloride disulfiram or a combination thereof.

17. The pharmaceutical composition according to claim 4, wherein the tumor cells are fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

18. The pharmaceutical composition according to claim 4, further comprising a cytotoxic agents selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus, deforolimus, paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydro-xy-3,5-dimethoxyphenyl]-5,5a,6,8,8a,-9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethy-1]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna.

19. A method of ameliorating one or more symptom of cancer in subject comprising the steps of:
providing a subject thought to be suffering from one or more symptom of cancer;
providing an effective amount of a cytotoxic agents and a disulfiram composition, a nitrosylating agent or both in a pharmaceutically acceptable carrier to inhibit an O⁶-methylguanine DNA methyltransferase and ameliorating one or more symptom of cancer.

20. The method according to claim 19 wherein the nitrosylating agent is nitroaspirin and the disulfiram composition is disulfiram, copper-chelated disulfiram, copper-chloride disulfiram or a combination thereof.

21. The method according to claim 19, wherein the cancer is cells selected from: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, brain cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

22. The method according to claim 19, further comprising the step of providing one or more cytotoxic agents selected from cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, doxorubicin heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunor-ubicin, rapamycin and its derivatives, sirolimus, temsirolimus, everolimus, zotarolimus, deforolimus, paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(-3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, BMS 188797, topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)-ethyl]-N-methylamino]ethyl]-5-[4-Hydro-xy-3,5-dimethoxyphenyl]-5,5a,6,8,8a,-9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethy-1]formamide, N-(2-(dimethylamino)ethyl)acrid-ine-4-carboxamide, 6-[[2-(dimethylamino)ethyl]amino]-3-hydroxy-7H-indeno[2-,1-c]quinolin-7-one, and dimesna.
